(19) United States
(12) Patent Application Publication
(10) Pub. No.: US 2021/0315604 A1
(43) Pub. Date: Oct. 14, 2021

(54) SYSTEMS AND METHODS FOR PROVIDING DISTAL EMBOLIC PROTECTION

(71) Applicant: BOARDS OF REGENTS OF THE UNIVERSITY OF TEXAS SYSTEM, Austin, TX (US)

(72) Inventor: Anand Prasad, Austin, TX (US)

(21) Appl. No.: 17/259,532

(22) PCT Filed: Jul. 11, 2019
(86) PCT No.: PCT/US2019/041497

§ 371 Date: Jan. 11, 2021

Related U.S. Application Data
(60) Provisional application No. 62/696,806, filed on Jul. 11, 2018.

(51) Int. Cl. A61B 17/3203; A61B 17/00; A61B 17/221

(52) U.S. Cl. CPC A61B 17/32037; A61B 17/00234; A61B 2017/00243; A61B 2017/00292; A61B 17/221

(57) ABSTRACT

In one embodiment, a system for providing a distal embolic protection including a distal embolic protection flow catheter configured for insertion into an artery, the catheter comprising one or more outlet apertures configured to eject fluid into the artery to drive embolic particles within the artery in a desired direction

### CROSS-REFERENCE TO RELATED APPLICATION

This application claims priority to co-pending U.S. Provisional Application Ser. No. 62/696,806, filed Jul. 11, 2018, which is hereby incorporated by reference herein in its entirety.

### BACKGROUND

Distal embolization (DE) is a feared complication of coronary, peripheral, and valvular procedures. Calcific, atherosclerotic, and thrombotic debris can be liberated as embolic particles during manipulation of surgical instruments and devices, inflation of balloons, deployment of stents, and implantation of percutaneous valves (e.g., transcatheter aortic valve replacement (TAVR) valves). Such particles can be macroscopic or microscopic and DE can be subclinical or manifest as a clinical event. Depending on the distal vascular bed, DE can result in stroke/transient ischemic attack (ITA), acute kidney injury (renal arterial embolization), or arterial bed embolization (tissue ischemia/pain).

Current techniques to reduce DE risk in the lower extremities include use of wire-based filters. Such devices use small (e.g., 0.014 in.) diameter wires that form baskets designed to capture debris within the vessel. Unfortunately, these filters exhibit various disadvantages, including vessel wall damage from the filter, entrapment risk in stents, incomplete capture of debris, difficulty in retrieval when full, need for an adequate anatomic landing zone, and added cost. For TAVR procedures, specific filter baskets have been designed and shown to capture particles, but have not reduced the incidence of stroke or TIA.

In view of the above discussion, it can be appreciated that it would be desirable to have a way to protect against DE other than by implanting a wire-based filter.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1A is a diagram that illustrates an example of providing distal embolic protection within the aortic arch with a first embodiment of a distal embolic protection flow catheter.

FIG. 1B is a diagram that illustrates an example of providing distal embolic protection within the aortic arch with a second embodiment of a distal embolic protection flow catheter.

FIG. 1C is a diagram that illustrates an example of providing distal embolic protection within the aortic arch with a third embodiment of a distal embolic protection flow catheter.

FIG. 2A is a diagram that illustrates an example of providing distal embolic protection within the abdominal aorta with a fourth embodiment of a distal embolic protection flow catheter.

FIG. 2B is a diagram that illustrates an example of providing distal embolic protection within the abdominal aorta with a fifth embodiment of a distal embolic protection flow catheter.

### DETAILED DESCRIPTION

As described above, it would be desirable to have a way to protect against distal embolization (DE) other than by implanting a wire-based filter. Disclosed herein are systems and methods designed for this purpose. In some embodiments, a system for providing distal embolic protection comprises a distal embolic protection flow catheter that is configured to eject an appropriate fluid, such as saline, within a vessel, such as an artery, for the purpose of driving embolic particles away from organs of interest. In some embodiments, the catheter comprises one or more outlet apertures through from which the fluid can be ejected in either a continuous or pulsatile manner. As an example, the catheter can be used to drive the particles farther along a primary vessel (e.g., aorta) so as to prevent the particles from entering a branch vessel (e.g., carotid artery) that leads to the organ (e.g., brain). In some embodiments, the catheter can additionally comprise inlet apertures that can be used to remove the particles from the vessel.

In the following disclosure, various specific embodiments are described. It is to be understood that those embodiments are example implementations of the disclosed inventions and that alternative embodiments are possible. Such alternative embodiments include hybrid embodiments that comprise aspects of different embodiments. All such embodiments are intended to fall within the scope of this disclosure.

As described above, a system and method for providing distal embolic protection implement a distal embolic protection flow catheter that can drive embolic particles away from an organ of interest. In some cases, the catheter can be used to divert particles away from the cerebral vessels (e.g., carotid and/or vertebral arteries) to prevent stroke or transient ischemic attack (TIA). In other cases, the catheter can be used to divert particles away from the renal arteries to prevent acute kidney injury (AKI). Generally speaking, the catheter creates a flow that is analogous to that of a fast moving river. Specifically, the flow generated by the catheter carries debris (embolic particles) away from side branches (branch vessels) and downstream along the “river” (primary vessel).

In addition to the catheter, the system and method can implement one or more other devices, such as a pump, which can be used to drive the fluid through the catheter and out one or more of its outlet apertures such that the fluid is injected into the vessel. In some embodiments, the one or more outlet apertures can comprise a nozzle that generates a jet of fluid that is particularly well-suited for diverting the embolic particles from the branch vessels.

In some embodiments, catheter can also be used to collect the particles so that they can be removed from the body. To that end, the catheter can include one or more inlet apertures through which fluid and particles can be drawn into the catheter and the system can include a suction device that creates a vacuum within the catheter. In such cases, the catheter can comprise a first internal lumen configured to supply fluid to the outlet aperture(s) and a second internal lumen configured to receive fluid/particles from the inlet aperture(s).

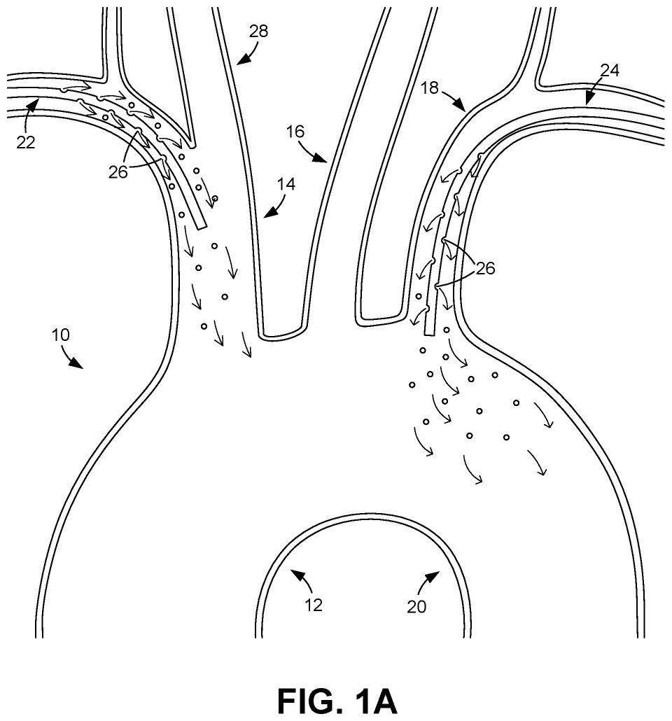

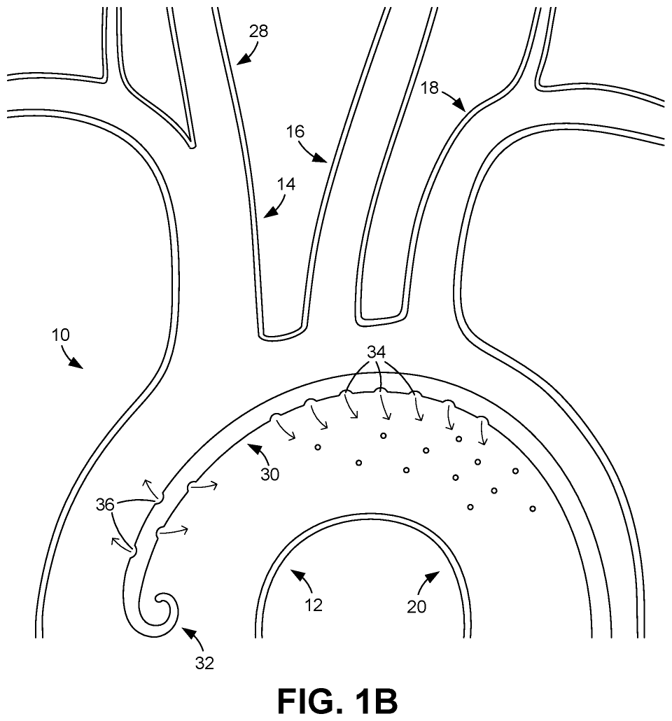

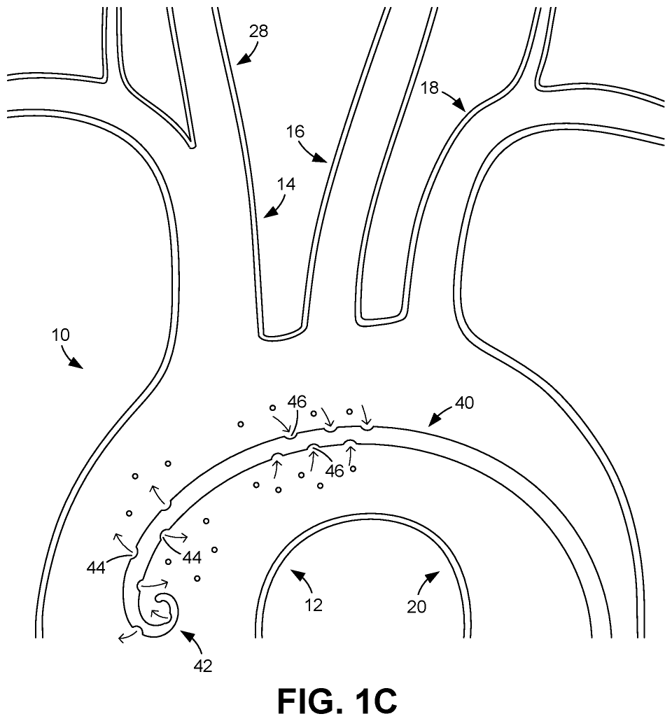

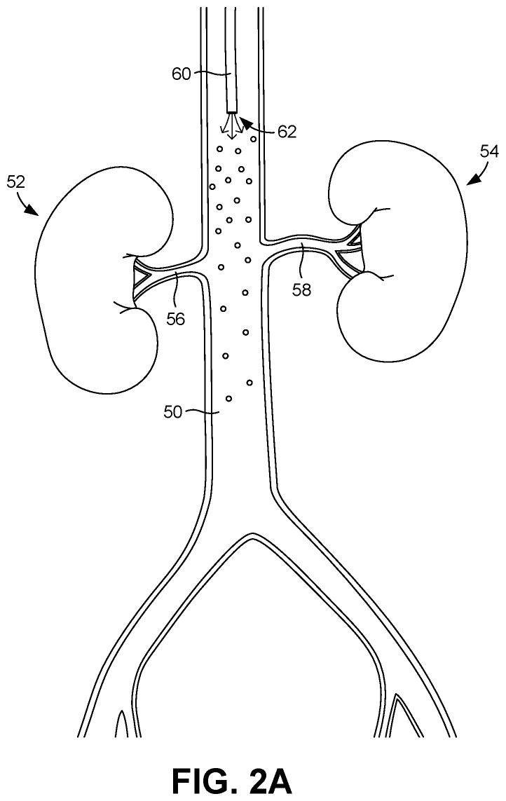

FIGS. 1A-1C illustrate distal embolic protection being provided within the ascending aortic arch using various embodiments of distal embolic protection flow catheters. Beginning with FIG. 1A, illustrated is the anatomy of the ascending aortic arch 10, which includes the ascending aorta 12, the innominate artery 14, the left common carotid artery 16, the left subclavian artery 18, and the descending aorta 20. As illustrated in the figure, a first distal embolic protection flow catheter 22 is positioned within the innominate artery 14 via radial artery access and a second distal embolic protection flow catheter 24 is positioned within the left subclavian artery 18 also via radial artery access. Each catheter 22, 24 can be made of a suitable flexible polymeric material and comprise a plurality of outlet apertures 26 extending through the side walls of the catheter near a distal tip of the catheter. In some embodiments, the apertures can be located on multiple sides of the catheter 22. Driving fluid (e.g., liquid), such as saline, can be ejected from the apertures 26. As noted above, the apertures 26 can, in some embodiments, be configured as nozzles that create jets of fluid. In addition, the catheters 22, 24 can either be provided with a distal opening at its distal tip or have a closed tip.

Irrespective of the nature of the outlet apertures 26, the fluid ejected from the apertures of the first distal embolic protection flow catheter 22 create a flow, represented by multiple arrows, that drives embolic particles, represented by small circles, that have reached the innominate artery 14 out of that artery and down to the ascending aorta 12 so that the particles cannot reach the brain via a branch artery, such as the right carotid artery 28. As noted above, the flow of fluid ejected from the catheter 22 can be continuous or pulsatile, depending upon how the fluid is supplied to the catheter. In similar manner, the fluid ejected from the apertures 26 of the second distal embolic protection flow catheter 24 creates a flow that drives embolic particles from the left subclavian artery 18 into the descending aorta 20. It can, therefore, be appreciated that the catheters 22, 24 are used to drive and maintain the particles within the aortic arch 10 so that they do not reach an organ of interest, such as the brain. In some embodiments, both catheters 22, 24 can be used together to create the flow. In other embodiments, the catheters 22, 24 can be used independent of each other.

FIG. 1B illustrates another example of distal embolic protection being provided within the ascending aortic arch 10. In this case, a single distal embolic protection flow catheter 30 is provided within the aortic arch 10, extending from the descending aorta 20 into the ascending aorta 12. In this embodiment, the catheter 30 is configured as a pigtail catheter having a distal end that forms a curled pigtail 32. As with the previous catheters 22, 24, the catheter 30 can be made of a suitable flexible polymeric material and comprise a plurality of outlet apertures 34 formed in the side walls of the catheter from which a driving fluid (e.g., saline) can be ejected. In the example of FIG. 1B, however, each of the apertures 34 is positioned on an inner side of a curvature of the catheter so as to direct embolic particles away from the branch arteries that extend upward from the aorta (in the orientation of the figure), which are located beyond the outer side of the catheter. In addition, the catheter 30 includes auxiliary outlet apertures 36 formed through the side walls of the catheter adjacent the pigtail 32 that can be used to eject contrast fluid into the vessel. When the catheter 30 comprises both such apertures 34, 36, the catheter can comprise two distinct inner lumens, one that supplies the driving fluid to the apertures 34, and one that supplies contrast fluid to the apertures 36.

FIG. 1C illustrates a further example of distal embolic protection being provided within the aortic arch 10. As in the case of FIG. 1B, a single distal embolic protection flow catheter 40 is provided within the aorta and extends from the descending aorta 20 into the ascending aorta 12. The catheter 40 is again configured as a pigtail catheter having a distal end that forms a curled pigtail 42. The catheter 40 comprises a plurality of outlet apertures 44 formed through the side walls of the catheter near and along the pigtail 42 from which a driving fluid can be ejected. In this embodiment, the fluid can be saline, contrast fluid, or both. In addition to the outlet apertures 44, inlet apertures 46 are formed through the side walls of the catheter 40 proximal of the outlet apertures that can be used to draw in embolic particles and/or contrast fluid for their removal from the vessel. When the catheter 40 comprises both such apertures 44, 46, the catheter can also comprise two distinct inner lumens, one that supplies fluid to the apertures 44, and one that receives fluid collected by the apertures 46.

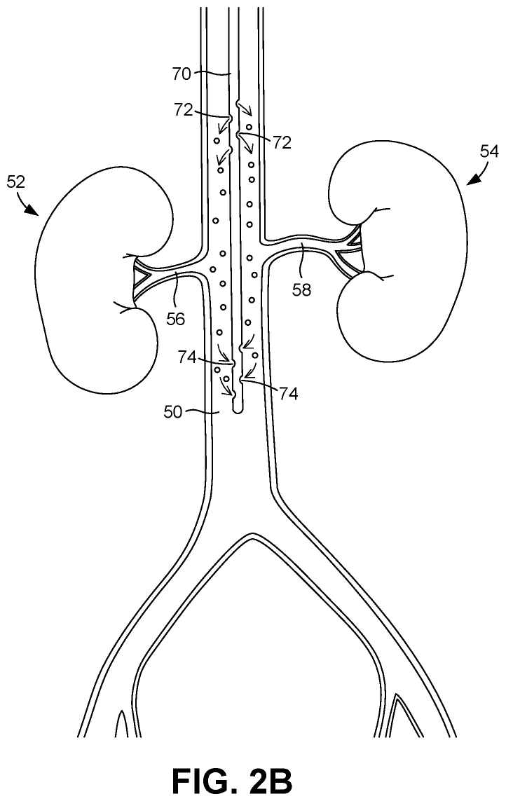

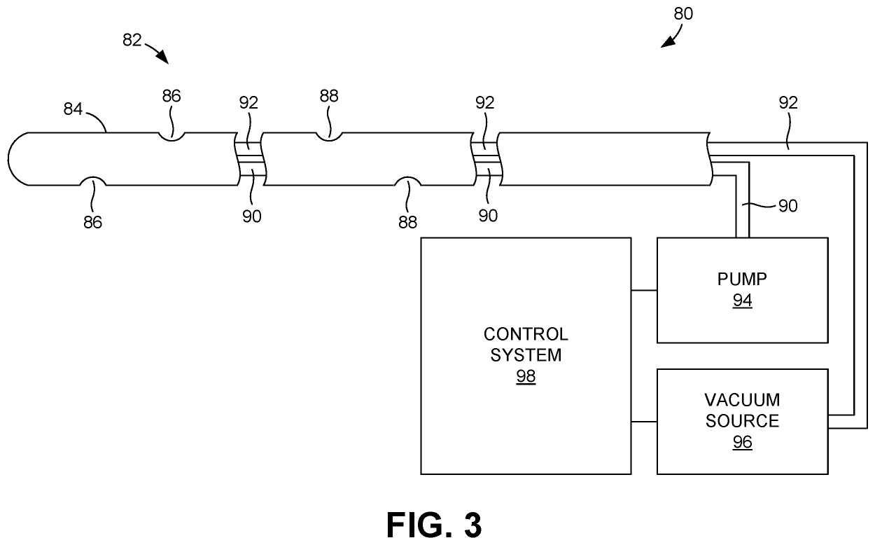

FIGS. 2A and 2B illustrate distal embolic protection being provided within the abdominal aorta using two different embodiments of distal embolic protection flow catheters. Beginning with FIG. 2A, illustrated is the abdominal aorta 50 and the right and left kidneys 52 and 54, which are placed in fluid communication with the aorta via right and left renal arteries 56 and 58. The figure depicts embolic particles that have traveled down the abdominal aorta 50 and adjacent the renal arteries 56, 58. With embolic protection, there is a risk of the particles passing through the renal arteries 56, 58 and becoming lodged within the kidneys 52, 54. A distal embolic protection flow catheter 60 is provided within the abdominal aorta 50 and protects the kidneys 52, 54 from these particles by ejecting fluid from a distal outlet aperture 62 provided at the distal tip of the catheter. The flow created by the ejected fluid drives the embolic particles past the renal arteries 56, 58 so that the particles do not reach the kidneys 52, 54. In addition, this flow may further draw particles out from the renal arteries 56, 58 to protect the kidneys 52, 54.

With reference next to FIG. 2B, a further distal embolic protection flow catheter 70 is shown provided in the abdominal aorta 50. In this embodiment, the catheter 70 comprises a plurality of outlet apertures 72 from which driving fluid can be ejected. In addition, the catheter 70 includes a plurality of inlet apertures 74 with which the embolic particles can be collected and removed. As before, the catheter 70 can comprise distinct inner lumens that are associated with apertures 72 and apertures 74, respectively.

As noted above, the one or more distal embolic protection flow catheters can comprise part of a system for providing distal embolic protection that includes other components. FIG. 3 illustrates an example of one such system 80. As shown in this figure, the system 80 comprises a distal embolic protection flow catheter 82. The catheter 82 includes a continuous outer wall 84 through which various apertures are formed. In the example of FIG. 3, these apertures include outlet apertures 86 from which a driving or contrast fluid (e.g., liquid) can be ejected and inlet apertures 88 through which fluid can be drawn into the catheter, such as blood and the ejected fluid, as well as particles suspended in that fluid. Extending through the catheter 82 are first and second inner lumens 90 and 92 that are in fluid communication with the outlet apertures 86 and the inlet apertures 88, respectively.

With further reference to FIG. 3, the system 80 also includes a pump 94, or other fluid driving means, that is in fluid communication with the first inner lumen 90 and a vacuum source 96 that is in fluid communication with the second inner lumen 92. As such, the pump 94 is in fluid communication with the outlet apertures 86 and the vacuum source 96 is in fluid communication with the inlet apertures 88. In addition, the pump 94 can be in fluid communication with a first reservoir (not shown) that contains fluid to be ejected from the catheter 82, and the vacuum source 96 can be in fluid communication with a second reservoir (not shown) that is configured to receive and contain the fluid drawn into the catheter.

Finally shown in FIG. 3 is a control system 98 that is configured to control operation of at least the pump 94 and the vacuum source 96. In particular, the control system 98 can operate the pump 94 to drive fluid through the first inner lumen 90 and out from the outlet apertures 86, and to operate the vacuum source 96 to drawn in fluid through the inlet apertures 92 and along the second inner lumen 92. In some embodiments, the control system 98 comprises a computer including a processor and memory, the memory storing a software program comprising computer-readable instructions that are configured to control at least the operation of the pump 94 and the vacuum source 96. In some embodiments, the control system 98 can further include one or more sensors that collect data that can be used by the control system as feedback to determine when and how to operate the pump 94 and the vacuum source 96.

Claimed are:

1. A system for providing distal embolic protection, the system comprising:
a distal embolic protection flow catheter configured for insertion into an artery, the catheter comprising one or more outlet apertures configured to eject fluid into the artery to drive embolic particles in the artery in a desired direction.

2. The system of claim 1, wherein the catheter comprises a plurality of outlet apertures.

3. The system of claim 1, wherein the one or more outlet apertures are located near a distal tip of the catheter.

4. The system of claim 1, wherein the one or more outlet apertures are located proximal of a distal tip of the catheter.

5. The system of claim 1, wherein the one or more outlet apertures are formed as nozzles that are configured to eject jets of fluid.

6. The system of claim 1, further comprising a pump that drives the fluid through the catheter and out from the outlet apertures.

7. The system of claim 1, wherein the distal embolic protection flow catheter further comprises one or more inlet apertures configured to collect fluid and embolic particles contained within the vessel.

8. The system of claim 7, further comprising a vacuum source that draws fluid through the inlet apertures.

9. A method for providing distal embolic protection, the method comprising:
inserting a distal embolic protection flow catheter into an artery of a patient;
delivering fluid through the catheter to outlet apertures of the catheter; and
ejecting the fluid from the outlet apertures so as to create a flow within the artery that carries embolic particles contained within blood within the artery in a desired direction.

10. The method of claim 9, wherein inserting a distal embolic protection flow catheter comprises inserting the catheter into the aortic arch.

11. The method of claim 9, wherein inserting a distal embolic protection flow catheter comprises inserting the catheter into a branch artery that extends from the aorta.

12. The method of claim 9, wherein delivering fluid comprises delivering saline.

13. The method of claim 9, wherein ejecting the fluid create a flow that carries the particles away from a branch artery that leads to a vital organ.

14. The method of claim 13, wherein the vital organ is the brain.

15. The method of claim 13, wherein the branch artery is a renal artery and the vital organ is a kidney.
